(19) United States
(12) Patent Application Publication
(10) Pub. No.: US 2021/0353792 A1
(43) Pub. Date: Nov. 18, 2021

(54) METHOD AND APPARATUS FOR DECONTAMINATING ARTICLES IN A STEAM STERILIZER AT LOW TEMPERATURE

(71) Applicant: American Sterilizer Company, Mentor, OH (US)

(72) Inventors: David F. McCall, Painesville, OH (US); Kenneth J. Klobusnik, Lake City, PA (US); Mark E. Chiffon, Concord Twp., OH (US)

(73) Assignee: American Sterilizer Company, Mentor, OH (US)

(21) Appl. No.: 17/319,662

(22) Filed: May 13, 2021

Related U.S. Application Data
(60) Provisional application No. 63/025,484, filed on May 15, 2020.

(51) Int. Cl. A61L 2/07; A61L 2/24

(52) U.S. Cl. CPC A61L 2/07; A61L 2/24; A61L 2202/20; A61L 2202/123; A61L 2202/122

(57) ABSTRACT

A method and apparatus for reprocessing decontaminating articles in a steam sterilizer at low temperatures and sub-atmospheric pressure. The method includes steps for operating the steam sterilizer such that it can safely and effectively decontaminate articles at temperatures below conventional steam sterilization processing temperatures. The steam sterilizer includes a low temperature operating mode that processes articles in a chamber at low temperatures and at sub-atmospheric pressure.

### RELATED APPLICATION

This application claims the benefit of U.S. Provisional Application No. 63/025,484, filed May 15, 2020, which is hereby fully incorporated herein by reference.

### FIELD OF THE INVENTION

The present invention relates generally to the field of decontamination, and more particularly to a method and apparatus for decontaminating articles in a steam sterilizer at a low temperature and sub-atmospheric pressure.

### BACKGROUND OF THE INVENTION

With the worldwide spread of highly infectious diseases, such as coronavirus COVID-19, a rapid surge in demand can arise for personal protection equipment (PPE). Consequently, shortages can quickly develop for PPEs, especially during an epidemic. Accordingly, there has been an urgent need to find both methods and apparatus that are capable of decontaminating PPEs for reuse.

Many PPEs are made of materials that are sensitive to high temperatures making them susceptible to damage during high temperature reprocessing. For example, one common type of PPE made of temperature sensitive material is a respiratory protective device, such as the N95 respirator. The N95 respirator forms a seal around a user's nose and mouth and filters airborne particles 0.3 microns or larger. Typically, N95 respirators are to be discarded after one-time use and not subject to reprocessing. While existing vaporized hydrogen peroxide sterilizers may offer suitable low temperature reprocessing, they typically have a small capacity and a small number of installed units. Therefore, such sterilizers have a limited value in the midst of an urgent health crisis. In the case of steam sterilizers, which have large capacity and are in widespread use, the articles being reprocessed are subject to the high processing temperatures of a conventional steam sterilization cycle (e.g., 121° C. (250° F.) to 135° C. (275° F.)), thereby causing damage to the PPE and limiting safe reuse. Under the ordinary high temperature processing provided by a steam sterilizer, it has been found that the structural integrity of N95 respirators can be damaged such that they no longer provide a proper seal on a user's face, thereby providing less effective protection when reused.

The prior art fails to teach adaptation of the operational control modes of an existing steam sterilization apparatus used to effect conventional high temperature steam sterilization so that said steam sterilization unit can also be used to reprocess temperature-sensitive articles (e.g., PPEs such as N95 respirators) to effect decontamination of such articles at reduced temperatures, e.g., as low as 50° C. As a result, the prior art fails to make an existing installed base of steam sterilization apparatus rapidly available to reprocess heat sensitive articles that may be in high demand.

In view of the foregoing, there is a need for adapting existing sterilization apparatus to operate in a manner in which it can decontaminate temperature sensitive PPE for safe reuse. The present invention addresses drawbacks in the prior art by providing an adaptation to existing steam sterilizers allowing the steam sterilizer to decontaminate articles that can be damaged by high processing temperatures.

### SUMMARY OF THE INVENTION

In accordance with the present invention, there is provided a method for operating a steam sterilizer for decontaminating articles in a chamber, said method comprising: (a) operating a first valve to control a fluid connection of the chamber to a source of steam in order to regulate a temperature in the chamber to a predetermined exposure temperature, said predetermined exposure temperature being in a range from 50° C. to 100° C.; (b) operating a second valve to control fluid connection of the chamber to a vacuum source to regulate a pressure in the chamber to a predetermined exposure pressure, wherein said predetermined exposure pressure being in a range from 0 inHg to 29.92 inHg (HgV); and (c) regulating the predetermined exposure temperature and the predetermined exposure pressure in the chamber for a predetermined exposure time in order to effect decontamination of the articles.

In accordance with another aspect of the present invention, there is provided a steam sterilizer comprising: a chamber for receiving articles for reprocessing; a jacket surrounding the chamber; a first valve controlling fluid connection of the chamber to a source of steam; a second valve controlling fluid connection of the chamber to a vacuum source; and a control system for operating in a decontamination mode, wherein temperature of the chamber is regulated to a predetermined exposure temperature and a predetermined exposure pressure for a predetermined exposure time to effect decontamination of the articles therein, wherein the predetermined exposure temperature is in a range from 50° C. (122° F.) to 100° C. (212° F.).

In accordance with yet another aspect of the present invention, there is provided a method of operating a steam sterilizer in a first operating mode wherein articles are sterilized under high temperature conditions at a temperature of at least 121° C. (250° F.) and above-atmospheric pressure, and in a second operating mode wherein articles are decontaminated under low temperature conditions at a temperature not greater than 100° C. (212° F.) and sub-atmospheric pressure.

An advantage of the present invention is the provision of a method for operating a steam sterilizer such that it can safely and effectively decontaminate articles at temperatures below conventional steam sterilization processing temperatures.

Another advantage of the present invention is the provision of a steam sterilizer that includes a low temperature operating mode that can safely and effectively decontaminate articles that can be damaged by exposure to the high temperatures of conventional steam sterilization.

Still another advantage of the present invention is the provision of a method of operating a steam sterilizer such that it is operable in both a high temperature sterilization mode at above-atmospheric pressure and a low temperature decontamination mode at sub-atmospheric pressure.

Still another advantage of the present invention is the provision of a steam sterilizer that includes a control system that allows an operator to switch between a high temperature sterilization mode at above-atmospheric pressure and a low temperature decontamination mode at sub-atmospheric pressure.

Still another advantage of the present invention is the provision of a method of operating a steam sterilizer in an operating cycle that cools or warms a treatment chamber in preparation for a low temperature sterilization mode at sub-atmospheric pressure.

Still another advantage of the present invention is the provision of a steam sterilizer that includes a control system having an operating cycle that cools or warms a treatment chamber in preparation for a low temperature sterilization mode at sub-atmospheric pressure.

Still another advantage of the present invention is the provision of a method of operating a steam sterilizer such that temperature variations are minimized throughout a treatment chamber under sub-atmospheric pressure.

Still another advantage of the present invention is the provision of a steam sterilizer that includes a control system that minimizes temperature variations throughout a treatment chamber under sub-atmospheric pressure.

Yet another advantage of the present invention is the provision of a method of operating a steam sterilizer such that temperature and pressure are independently controlled during a low temperature decontamination at sub-atmospheric pressure.

Yet another advantage of the present invention is the provision of a steam sterilizer that include a control system for independent control of temperature and pressure during a low temperature decontamination at sub-atmospheric pressure

These and other advantages will become apparent from the following description of illustrated embodiments taken together with the accompanying drawings and the appended claims.

### BRIEF DESCRIPTION OF THE DRAWINGS

The invention may take physical form in certain parts and arrangement of parts, an embodiment of which will be described in detail in the specification and illustrated in the accompanying drawings which form a part hereof, and wherein:

FIG. 1 is a schematic representation of an exemplary steam sterilizer as adapted for use in accordance with the present invention;

FIG. 2 illustrates marking of a respirator prior to reprocessing in the steam sterilizer;

FIG. 3 illustrates respirators placed in pouches;

FIG. 4 illustrates pouched respirators located on a loading cart rack;

FIG. 5 illustrates unpouched respirators loosely nested in an instrument tray;

FIG. 6 illustrates unpouched respirators loaded flat on an instrument tray;

FIG. 7 illustrates a touchscreen display for selection of a DECON cycle;

FIG. 8 illustrates a touchscreen display for selection of a WARM/COOL cycle;

FIG. 9 illustrates a touchscreen display for initiating a print mode;

FIG. 10 illustrates an example printed record for a completed DECON cycle; and

FIG. 11 shows a flow diagram of an operational control scheme for the steam sterilizer, in accordance with an embodiment of the present invention.

### DETAILED DESCRIPTION OF THE INVENTION

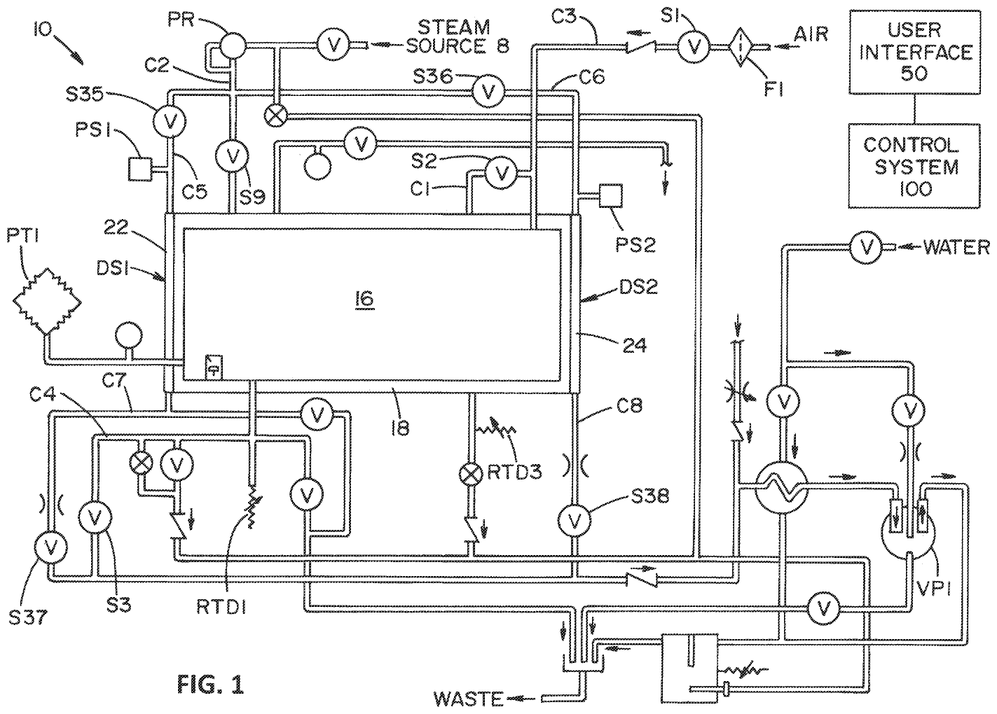

Referring now to the drawings wherein the showings are for the purposes of illustrating an embodiment of the invention only and not for the purposes of limiting same, FIG. 1 is a schematic representation of an exemplary steam sterilizer 10, as used in connection with the method of the present invention. It should be appreciated that sterilizer 10 is shown solely for the purpose of illustrating an embodiment of the present invention, and not for limiting same. Accordingly, it is contemplated that the present invention may be implemented in connection with steam sterilizers different from illustrated sterilizer 10. Examples of commercially available steam sterilizers include the AMSCO® 400 Series steam sterilizer and the AMSCO® Century® steam sterilizer from STERIS Corporation.

Furthermore, while the present invention is described herein with particular reference to the decontamination of a respiratory protective device, and more specifically to an N95 respirator, it should be understood that the present invention is suitable for use in the decontamination of a wide range of different types of PPEs, as well as other temperature sensitive articles, where lower processing temperatures are needed to prevent damage. References herein to respiratory protective devices and N95 respirators are not intended to limit the scope of the present invention solely to the decontamination of such articles.

With regard to the use of inHg as a unit of measure for pressure, it should be noted that pressure levels are referenced herein as “Hg gauge” (HgV), where the scale starts at 0 inHg (atmospheric pressure) and increases up to 29.92 inHg (perfect vacuum).

Sterilizer 10 is generally comprised of a control system 100; a sterilization chamber 16; a double wall jacket 18 that surrounds chamber 16; a loading door 22 controlled by control system 100 and associated with a door seal DS1; an unloading door 24 controlled by control system 100 and associated with a door seal DS2; a plurality of conduits; a plurality of valves (e.g., solenoid valves) controlled by control system 100; a plurality of sensors providing signals to control system 100; and a vacuum pump VP1 (or other vacuum source) controlled by control system 100. Each door seal DS1, DS2 has a respective associated door seal pressure switch PS1, PS2. It should be appreciated that alternative embodiments of exemplary sterilizer 10 may have only a single door (e.g., the illustrated loading door 22) for both loading and unloading. In the illustrated schematic of FIG. 1, the optional second door is unloading door 24 associated with door seal DS2. Furthermore, in alternative embodiments door seals DS1 and DS2 may take the form of a compression gasket seal or a seal activated by compressed air or other means. Sterilizer 10 is also comprised of a user interface 50 that includes a touchscreen display 52, an audio output for providing audible signals, and a printer.

The plurality of conduits include an interconnect conduit C1 in fluid communication with both jacket 18 and chamber 16 (i.e., fluidly connecting jacket 18 to chamber 16) and having a steam-to-chamber valve S2 disposed therein; a steam inlet conduit C2 in fluid communication with a steam source 8 (e.g., a central boiler or a dedicated steam generator that provides steam at a pressure in the range of 50-80 PSIG at temperature in the range of 297° F. (147.2° C.) to 325° F. (162.8° C.)) and jacket 18, said steam inlet conduit C2 having a steam-to-jacket valve S9 and steam pressure regulator PR disposed therein; an air inlet conduit C3 in fluid communication with interconnect conduit C1 and having an air valve 51 and an air filter μl disposed therein; and a drain conduit C4 in fluid communication with chamber 16 and having a chamber drain valve S3 disposed therein. Vacuum pump VP1 is also disposed in drain conduit C4. Each door seal DS1, DS2 respectively includes a pair of associated door seal conduits C5, C7 and C6, C8. The first conduit C5, C6 respectively associated with each door seal DS1, DS2 has a seal valve S35, S36 disposed therein and provides fluid communication between door seal DS1, DS2 and steam inlet conduit C2. The second conduit C7, C8 respectively associated with each door seal DS1, DS2 has a seal exhaust valve S37, S38 disposed therein and provides fluid communication between door seal DS1, DS2 and drain conduit C4.

The plurality of sensors include a chamber temperature sensor RTD1 for measuring the temperature in chamber 16, a jacket temperature sensor RTD3 for measuring the temperature in jacket 18, and a chamber pressure transducer PT1 for measuring the pressure inside chamber 16. In the illustrated embodiment of exemplary sterilizer 10, temperature sensors RTD1 and RTD3 take the form of a resistance temperature detector (RTD) located proximate to drain conduit C4.

Control system 100 receives signals from the sensors RTD1, RTD3, and PT1; receives signals from pressure switches PS1, PS2 respectively disposed in door seal conduits C5, C6; and transmits signals to the valves 51, S2, S3, S9, vacuum pump VP1, and doors 22, 24. Control system 100 is programmed to control sterilizer 10 according to various system operating cycles, which are described in detail below.

In accordance with an illustrated embodiment of the present invention, sterilizer 10 is programmed to operate in a low temperature decontamination mode wherein an article located in chamber 16 is exposed to a temperature in the range of about 63° C. (145.4° F.) to 73° C. (163.4° F.)) and a sub-atmospheric pressure in the range of 10-24 inHg, for an exposure time of 30 minutes. This low temperature decontamination mode is referred to a DECON cycle. Sterilizer 10 is also programmed to operate in a warm-up or cool-down mode where the temperature within chamber 16 is increased or decreased to prepare chamber 16 for a DECON cycle. This warm-up/cool-down mode is referred to as a WARM/COOL cycle. In the illustrated embodiment, sterilizer 10 is also operable in a standard sterilization mode where an article is exposed to high temperatures in the range of about 250° F. (121.1° C.) to 275° F. (135° C.). For instance, in a standard sterilization mode, sterilizer 10 may be programmed with an exposure temperature that is selectable from temperatures of 250° F. (121.1° C.), 270° F. (132.2° C.), or 275° F. (135° C.). This standard sterilization mode is referred to as a STERILIZE cycle. Reference is also made herein to a Daily Air Removal Test (DART), which is a test run, by sterilizer 10 to test the adequacy of air removal from chamber 16 and load.

The DECON cycle of the present invention is a decontamination process, and is not a sterilization process, as provided by the STERILIZE cycle. Therefore, it should be understood that the decontamination process of the DECON cycle does not inactivate as many organisms as the sterilization process of the STERILIZE cycle. However, the decontamination provided by the DECON cycle is recognized as being adequate for destroying many harmful bioburdens. As indicated above, one important advantage provided by the DECON cycle is that the low processing temperature allows temperature sensitive articles, such as respirators, masks, and other disposable PPEs, to be decontaminated for reuse without damage.

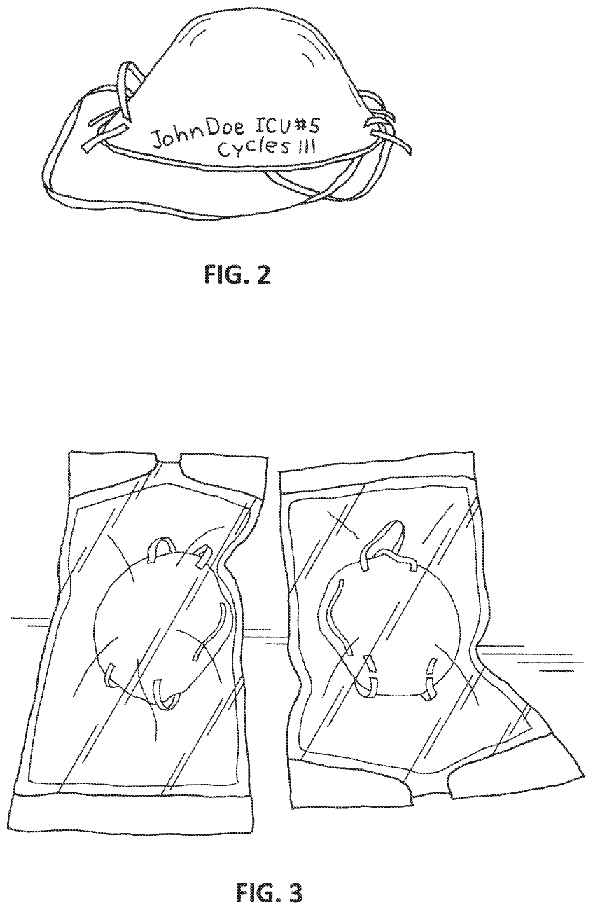

Prior to the reprocessing of PPEs, such as N95 respirators, it may be advantageous to mark the PPEs with information related to that PPE. For example, each PPE may be labeled (e.g., with a permanent marker) with the user's name, identifiers, and a numeric indication of the number of DECON cycles for the PPE (e.g., see FIG. 2 for marking of an N95 respirator). Alternatively, a bar code or RFID tag may be applied to each PPE to provide identification information, thereby facilitating handling and tracking of the PPEs. It should be appreciated that depending upon the type of PPE, it may be necessary to discard the PPE after a predetermined number of DECON cycles (e.g., 10 decontamination cycles for N95 respirators).

The DECON cycle of the present invention will now be described in detail according to an embodiment of the present invention as implemented in the operation of sterilizer 10 for the decontamination of N95 respirators. The DECON cycle includes a door seal phase, a conditioning phase, an exposure phase, a drying phase, and a seal retraction phase.

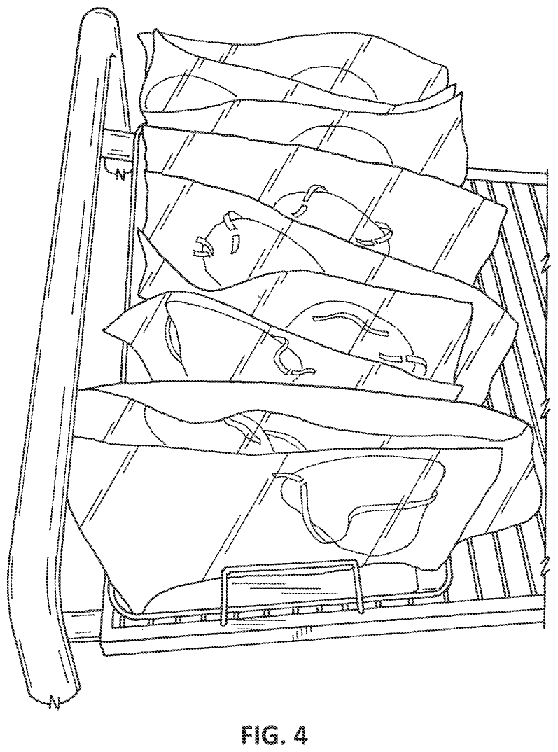

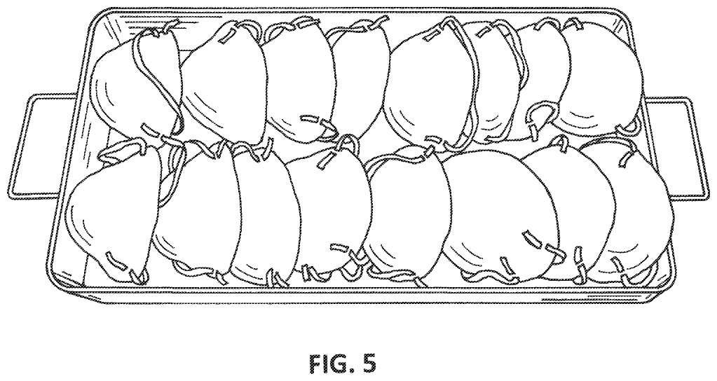

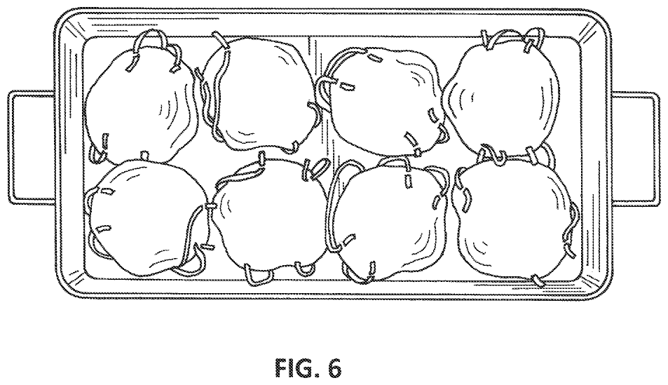

Before initiating the DECON cycle, N95 respirators are prepared for loading into sterilizer 10 by inspecting the respirators for any visible damage and/or excessive soil (i.e., blood, dried sputum, makeup, other soil). Respirators that are damaged or contain visible soil should be discarded. In an illustrated embodiment, respirators suitable for decontamination are placed in a pouch (e.g., self-seal pouches designed to permit moist heat transfer into the pouch), as seen in FIG. 3, and placed onto a loading cart rack (FIG. 4), a shelf, or an instrument tray. In accordance with an alternative embodiment, each respirator is placed unpouched directly on a loading cart rack, shelf, or instrument tray, either loosely nested (FIG. 5) or flat (FIG. 6). To facilitate proper decontamination, the respirators should not be stacked. After these preliminary preparation steps are completed, chamber 16 is loaded with the respirators and loading door 22 is closed and locked.

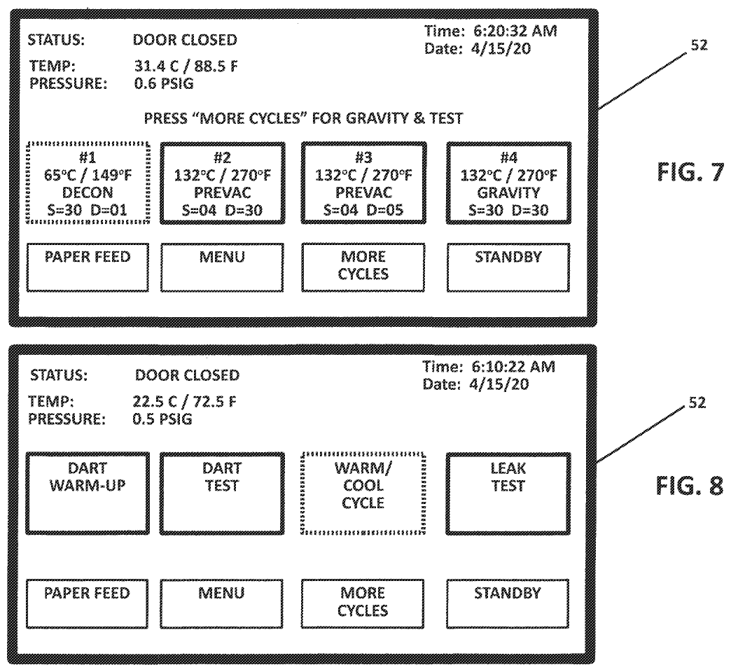

After closing and locking all doors 22, 24, an operator initiates a DECON cycle by selecting the DECON cycle on touchscreen 52 of user interface 50 (FIG. 7). With initiation of the DECON cycle, the door seal phase begins. In the door seal phase, control system 100 energizes (i.e., opens) seal valves S35, S36 and seal exhaust valves S37, S38 to purge door seals DS1, DS2, and thereafter seal exhaust valves S37, S38 are deenergized (i.e., closed) to seal the doors. Once the door seal pressure rises above a setpoint pressure for door seal pressure switches PS1, PS2 and stabilizes, the conditioning phase of the DECON cycle begins. In the illustrated embodiment, stabilization time for the door seal is about 15 seconds. It should be noted that chamber drain valve S3 is closed during the door seal phase to avoid exposing the chamber temperature sensor RTD1 to heat during purging of the door seals DS1, DS2.

In accordance with an alternative embodiment, the door seal phase is omitted. Accordingly, door seals DS1, DS2 are not pressurized as described above. In this regard, a compression door seal may be advantageous in controlling at low temperatures because no additional heat is applied to pressurize door seals DS1, DS2.

The conditioning phase follows the door seal phase. In the conditioning phase, steam-to-chamber valve S2 is energized to fluidly connect jacket 18 and chamber 16, and air valve S1 is closed. Vacuum pump VP1 is energized along with the chamber drain valve S3, thereby reducing the chamber and jacket pressure. Control system 100 pulses steam-to-jacket valve S9 to pulse steam into jacket 18 and chamber 16 where the temperature will rise in both. Pulsing only occurs when (i) the jacket temperature (as indicated by jacket temperature sensor RTD3) is below an exposure temperature (e.g., 65° C.) plus a jacket overdrive (5° C.) and (ii) the chamber temperature (as indicated by chamber temperature sensor RTD1) is below a control temperature (e.g., 66.5° C.). Vacuum pump VP1 continues to run. Chamber drain valve S3 remains energized (i.e., open) until chamber/jacket vacuum level reaches a sub-atmospheric pressure of 22 inHg as measured by chamber pressure transducer PT1. At this stage, control system 100 controls the chamber/jacket vacuum level by opening/closing chamber drain valve S3 as needed. Vacuum pump VP1 continues to operate for the entire exposure phase that follows the conditioning phase. Chamber temperature sensor RTD1 continues to measure an increasing temperature until the exposure temperature (65° C.) is detected. After a delay of approximately 20 seconds, control system 100 checks for a minimum vacuum level that is dependent on altitude (15 inHg from 0 to 1000 ft). Once the minimum vacuum level is reached, the exposure phase begins.

During the exposure phase, control system 100 continues to pulse steam-to-jacket valve S9 to control the temperature inside the chamber and jacket at a predetermined exposure temperature based on the temperature measurement provided by chamber temperature sensor RTD1. Control system 100 also opens and closes chamber drain valve S3 as needed to maintain the chamber vacuum pressure at a predetermined exposure pressure during the exposure phase. In the illustrated embodiment, decontamination is effected during the exposure phase by exposing the respirators in chamber 16 to an exposure temperature of 65° C. (149° F.), at an exposure pressure of 22 inHg, for a decontamination exposure time of 30 minutes. The selected exposure temperature and pressure maintain the steam in chamber 16 in a saturated condition. However, it should be appreciated that the exposure temperature (chamber temperature setpoint), exposure pressure (chamber pressure setpoint at sea level), and exposure time (amount of time the articles in chamber 16 are exposed to the exposure temperature/exposure pressure) may vary from the values mentioned above depending upon the temperature sensitive properties of the article being decontaminated and the level of decontamination desired. For instance, the exposure temperature may be in the range of about 63° C. (145.4° F.) to 73° C. (163.4° F.), the exposure pressure may be in the range of about 10 inHg to 24 inHg, and the exposure time may be greater than or less than 30 minutes. It is further contemplated that based upon the temperature sensitive properties of the article and the desired level of decontamination, the exposure temperature may range from about 50° C. (122° F.) to 100° C. (212° F.) and the exposure pressure may range from 0 inHg to 29.92 inHg, with an exposure time appropriate to prevent damage to the temperature sensitive article and effect the desired level of decontamination. In accordance with the present invention, the exposure temperature and exposure pressure are selected so that the article being decontaminated is exposed to saturated steam (i.e., steam that has not been heated above the boiling point for its pressure). Temperature and pressure values for saturated steam are readily available from saturated steam tables.

The exposure phase is followed by the drying phase for drying the articles in chamber 16. In the drying phase, the steam-to-chamber valve S2 is closed. Chamber drain valve S3 is opened and vacuum pump VP1 is on. The pressure inside chamber 16 is reduced during this time. Control system 100 operates steam-to-jacket valve S9 to control the jacket temperature at 62.2° C. A vacuum is pulled in chamber 16 for a set time of 1 minute. After 1 minute, chamber drain valve S3 is closed and vacuum pump VP1 is turned off. Air valve S1 is opened to draw filtered air into chamber 16, thereby causing the pressure in chamber 16 to rise to atmospheric pressure (0 inHg).

The seal retraction phase follows the drying phase. In the seal retraction phase, seal valves S35, S36 are deenergized. Vacuum pump VP1 and the seal exhaust valves S37, S38 are energized. The seal pressure is reduced below the setpoint pressure for door seal pressure switches PS1, PS2. The seal pressure reduction continues until the door seals DS1, DS2 are fully retracted. Thereafter, vacuum pump VP1 is deenergized.

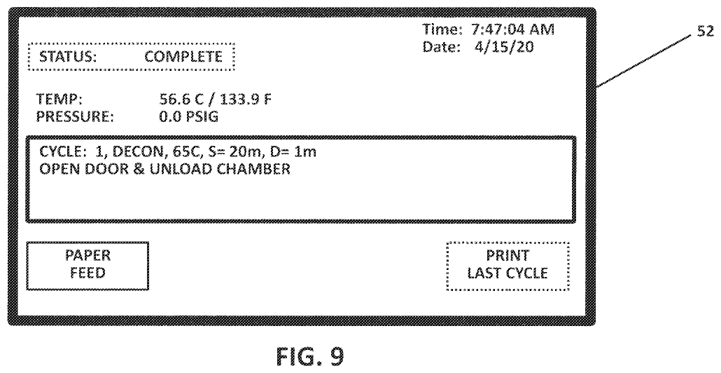

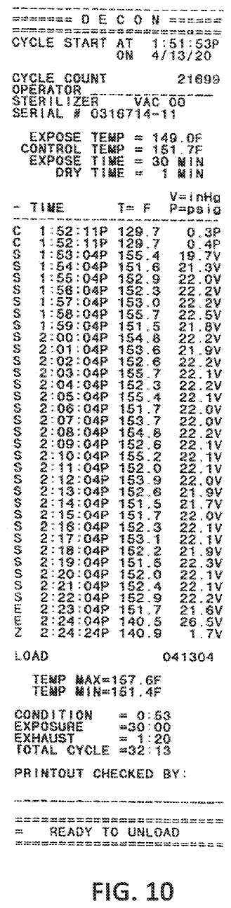

The DECON cycle is completed at the end of the seal retraction phase. In the illustrated embodiment, user interface 50 provides an audible tone indicating completion of the DECON cycle and that doors 22, 24 can be opened. Control system 100 controls the jacket temperature at 62.2° C. (144° F.) following completion of the DECON cycle and opening of unloading door 24. The results of the DECON cycle may be printed by the operator initiating a print mode on touchscreen 52 of user interface 50 (FIG. 9). Alternatively, control system 100 may be programmed to automatically print the results of the DECON cycle immediately following its completion. An example of a printed record for a completed DECON cycle is shown in FIG. 10. Following completion of the DECON cycle, the reprocessed respirators are removed from sterilizer 10 and loaded into disinfected trays or containers and placed in a closed case cart or trolley for transport and return to users

In accordance with the present invention, there is also provided a WARM/COOL cycle which is run with an empty chamber 16. However, if a loading cart rack is used, then it may be loaded into chamber 16 before initiating the WARM/COOL cycle in order to warm the loading cart rack and stabilize its temperature. The WARM/COOL cycle prepares sterilizer 10 to run a DECON cycle following the running of a standard high temperature STERILIZE cycle or following “power up” of sterilizer 10. It should be appreciated that the WARM/COOL cycle of the present invention allows sterilizer 10 to conveniently and efficiently operate in both high and low temperature decontamination modes. Under conditions where the sterilizer is too cool (e.g., jacket temperature is detected as being less than 70° C.), then a WARM/COOL cycle is run with a warming phase to warm up chamber 16 in preparation for the DECON cycle. Under conditions where the sterilizer is too warm (e.g., the jacket temperature is detected as being at or above 70° C.), then a WARM/COOL cycle is run with a cooling phase to cool down chamber 16 in preparation for the DECON cycle. The warming and cooling phases are described in detail below. Touchscreen 52 of user interface 50 instructs the operator that a WARM/COOL cycle is necessary to initiate.

After closing and locking the doors, an operator activates the WARM/COOL cycle by selecting WARM/COOL cycle on touchscreen 52 of user interface 50 (FIG. 8). The WARM/COOL cycle includes a door seal phase, a chamber temperature evaluation phase, a cooling or warming phase depending upon conditions, a drying phase, and a seal retraction phase. Upon selection of a WARM/COOL cycle, control system 100 lowers the jacket control temperature to control at the exposure temperature (65° C./149° F.) minus 2.8° C./5° F. Accordingly, control system 100 will deenergize steam-to-jacket valve S9 if jacket 18 was controlling at high temperature, but will also allow chamber 16 to continue warming if it is cold.

In the door seal phase, control system 100 energizes seal valves S35, S36 and seal exhaust valves S37, S38 to purge door seals DS1, DS2, and thereafter seal exhaust valves S37, S38 are deenergized to seal the doors. Once the door seal pressure rises above the setpoint pressure for door seal pressure switches PS1, PS2 and stabilizes, the chamber temperature evaluation phase of the WARM/COOL cycle will start. In the illustrated embodiment, stabilization time for the door seal is approximately 15 seconds.

The chamber temperature evaluation phase follows the door seal phase. In the chamber temperature evaluation phase, steam-to-chamber valve S2 is energized to fluidly connected jacket 18 and chamber 16, and air valve 51 is closed. Vacuum pump VP1 is energized (i.e. turned on) and chamber drain valve S3 is energized (i.e., opened), thereby reducing the chamber and jacket pressure. Steam-to-jacket valve S9 is deenergized (i.e., closed) to turn off the steam supply to jacket 18. If the jacket or chamber temperatures, as measured by temperature sensors RTD1 and RTD3, is at or above a cutoff temperature (e.g., 70° C.), then a cooling phase (described below) begins. Otherwise, the chamber temperature evaluation phase continues with a delay (e.g., 20 seconds) to check for latent heat in chamber 16 causing an increase in the measured temperature in chamber 16. If the chamber temperature is at or above the cutoff temperature after the delay, then the cooling phase begins. Otherwise, a warming phase (described below) begins.

In the cooling phase, control system 100 checks for a minimum vacuum level that is dependent on altitude (15 inHg from 0 to 1000 ft). The cooling phase continues once this vacuum level is reached with steam-to-jacket valve S9 deenergized (i.e., closed). In the cooling phase, steam is exhausted from the jacket to assist in cooling down sterilizer 10 in preparation to run the DECON cycle.

In the warming phase, steam-to-jacket valve S9 is controlled to pulse steam into jacket 18 and chamber 16, thereby causing the temperature to increase in both jacket 18 and chamber 16. Pulsing only occurs when (i) the jacket temperature, as measured by temperature sensor RTD3, is below the exposure temperature for the DECON cycle (e.g., 65° C.) plus the jacket overdrive (5° C.) and (ii) the chamber temperature, as measured by temperature sensor RTD1 is below the control temperature (e.g., 66.5° C.). Chamber drain valve S3 remains energized until the chamber/jacket vacuum level reaches 22 inHg, as measured by the chamber pressure transducer PT1. At this point, control system 100 controls the vacuum level by opening and closing chamber drain valve S3, as needed. The chamber temperature sensor RTD1 continues to measure a rising temperature until the exposure temperature for the DECON cycle (e.g., 65° C.) is reached. Control system 100 immediately starts checking for a minimum vacuum level that is dependent on altitude (15 inHg from 0 to 1000 ft). Once the minimum vacuum level is reached, the warming phase will continue for 30 minutes to stabilize the temperature throughout chamber 16. This concludes the warming phase.

After the cooling or warming phase is completed, a drying phase begins for drying chamber 16. In the drying phase, the steam-to-chamber valve S2 is closed. Chamber drain valve S3 is opened and vacuum pump VP1 is on. The pressure inside chamber 16 is reduced during this time. Control system 100 operates steam-to-jacket valve S9 to control the jacket temperature at 62.2° C. if warming chamber 16. A vacuum is pulled in chamber 16 for a set time of 1 minute. After 1 minute, chamber drain valve S3 is closed and vacuum pump VP1 is turned off. Air valve S1 is opened to draw filtered air into chamber 16, thereby causing the pressure in chamber 16 to rise to atmospheric pressure.

A seal retraction phase follows the drying phase. In the seal retraction phase, seal valves S35, S36 are deenergized. Vacuum pump VP1 and seal exhaust valves S37, S38 are energized. The seal pressure is reduced below the setpoint pressure for door seal pressure switches PS1, PS2. At this time, vacuum pump VP1 is deenergized.

After completion of drying and seal retraction phases that follow a cooling phase, a tone will sound and the operator is required to open door 22 or 24 to chamber 16 for processing to continue. Once the door to chamber 16 is opened, a 1-hour timer counts down the required wait time. At the end of the 1-hour, control system 100 controls the jacket temperature to 65° C./149° F. minus 2.8° C./5° F.

After completion of drying and seal retraction phases that follow a warming phase, a tone will sound and door 22 or 24 for accessing chamber 16 can be opened by the operator. Control system 100 controls the jacket temperature at 62.2° C. following completion of the WARM/COOL cycle and opening of the door to chamber 16.

It should be noted that control system 100 detects when the jacket temperature is set too low (e.g., 50° C. (122° F.) for a low temperature DECON cycle and 115.6° C. (240° F.) for high temperature STERILIZE cycles) and forces a WARM/COOL cycle or a DART WARM UP cycle to be run in order to stabilize the temperature in chamber 16 before a low temperature DECON cycle or a high temperature STERILIZE cycle can be run. Cycling power out of cycle causes control system 100 to revert to low temperature control for DECON cycles due to the long time required to cool the sterilizer.

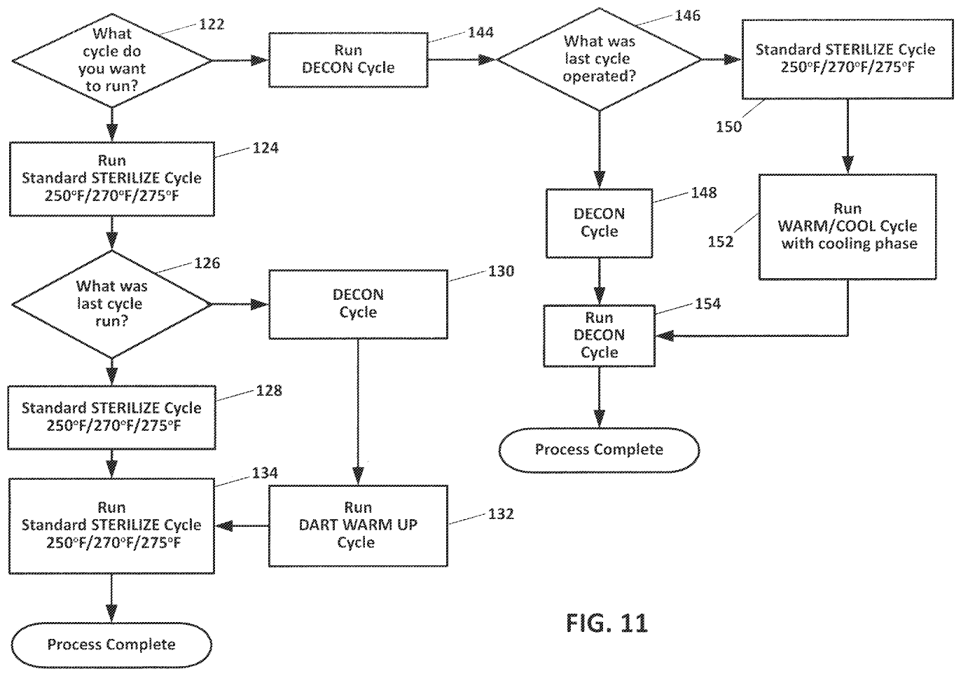

FIG. 11 shows a flow diagram of an operational control scheme for steam sterilizer 10, including steps for preparing sterilizer 10 to run a DECON cycle according to an embodiment of the present invention.

Initially, the operator selects a cycle to be run using touchscreen 52 (step 122). If the operator selects a standard STERILIZE cycle (step 124), then control system 100 control system 100 determines what was the last cycle run by sterilizer 10 (step 126). If the last cycle run was a standard STERILIZE cycle (step 128), then control system 100 runs a standard STERILIZE cycle at the desired temperature (step 134). If the last cycle run was a DECON cycle (step 130), then control system 100 runs a DART WARM UP cycle (step 132). In the illustrated embodiment, a DART WARM UP cycle is run with an empty chamber. When the DART WARM UP cycle is completed, then control system 100 runs a standard STERILIZE cycle at the selected temperature (step 134).

If the operator selects a DECON cycle (step 144) in response to step 122, then control system 100 determines what was the last cycle run by sterilizer 10 (step 146). If the last cycle run by sterilizer 10 was a DECON cycle (step 148), then control system 100 starts a new DECON cycle (step 154). If the last cycle run by sterilizer 10 was a standard STERILIZE cycle, then control system 100 runs a WARM/COOL cycle with a cooling phase (step 152). Chamber 16 may be allowed to cool for about 1 to 1.5 hours. After the WARM/COOL cycle is completed, control system 100 runs a DECON cycle (step 154).

In accordance with the illustrated embodiment, upon “power up” of sterilizer 10, a DART WARM UP cycle is run prior to running a STERILIZE cycle, and a WARM/COOL cycle is run prior to running a DECON cycle.

It should be appreciated that jacket 18 can get condensate build-up over time if only DECON cycles are run. This build-up of condensate results from a lack of pressure to blow the condensate out through a steam trap. Therefore, it is contemplated that the operational control scheme for steam sterilizer 10 may be modified to include steps for control system 100 detecting the presence of condensate and blowing out condensate via a steam trap. The presence of condensate may be indicated by (i) the number of DECON cycles run in a row, (ii) the amount of time jacket 18 is controlled at a low temperature, (iii) the jacket temperature response, or (iv) a combination of the foregoing. In an alternative embodiment, jacket 18 is sufficiently pressurized to blow out the condensate without having to run a WARM/COOL cycle again.

Furthermore, it is contemplated that temperature control of the DECON cycle could be made tighter by use of one or more load probes (i.e., temperature sensing probes) located inside chamber 16. A tip of the load probe can be placed within the load being sterilized.

In addition, it is contemplated that steam sterilizer 10 may be configured in an alternative embodiment to operate only in a low temperature DECON cycle (i.e., no high temperature STERILIZED cycle). Accordingly, in this embodiment, chamber temperature sensor RTD1 and jacket temperature sensor RTD3 may be placed in different locations that allow better sensing of the chamber and jacket temperatures with little or no steam flow to the drain through drain conduit C4. In the embodiment described above, the locations of chamber temperature sensor RTD1 and jacket temperature sensor RTD3 are selected to optimize high temperature STERILIZE cycles.

The foregoing describes specific embodiment(s) of the present invention. It should be appreciated that these embodiments are described for purposes of illustration only. Other modifications and alterations will occur to others upon their reading and understanding of the specification. As indicated above, the present invention is not limited to decontamination of N95 respirators or PPEs, but also finds utility in decontamination of other types of temperature-sensitive articles which that can be damaged by exposure to high temperatures. Moreover, the exposure temperature, exposure pressure, and exposure time may vary from those values disclosed in connection with the illustrated embodiment. It is intended that all such modifications and alterations be included insofar as they come within the scope of the invention as claimed or the equivalents thereof.

Having described the invention, the following is claimed:

1. A method for operating a steam sterilizer for decontaminating articles in a chamber, said method comprising:
operating a first valve to control a fluid connection of the chamber to a source of steam in order to regulate a temperature in the chamber to a predetermined exposure temperature, said predetermined exposure temperature being in a range from 50° C. to 100° C.;
operating a second valve to control fluid connection of the chamber to a vacuum source to regulate a pressure in the chamber to a predetermined exposure pressure, wherein said predetermined exposure pressure being in a range from 0 inHg to 29.92 inHg (HgV); and
regulating the predetermined exposure temperature and the predetermined exposure pressure in the chamber for a predetermined exposure time in order to effect decontamination of the articles.

2. The method according to claim 1, wherein the method further comprises:
determining if the temperature in the chamber is at or above a cutoff temperature; and
if the temperature in the chamber is at or above the cutoff temperature, then operating the sterilizer in a cooling phase to reduce the temperature in the chamber prior to decontamination of the articles.

3. The method according to claim 2, wherein the cutoff temperature is about 70° C.

4. The method according to claim 1, wherein the method further comprises:
determining if the temperature in the chamber is at or above a cutoff temperature; and
if the temperature in the chamber is below the cutoff temperature, then operating the sterilizer in a heating phase to increase the temperature in the chamber prior to decontamination of the articles.

5. The method according to claim 4, wherein the temperature in the chamber is increased in the heating phase by pulsing steam into the chamber.

6. The method according to claim 1, wherein the predetermined exposure time is about 30 minutes.

7. The method according to claim 1, wherein said articles include personal protection equipment (PPE) made of material sensitive to high processing temperatures in a range of 121° C. (250° F.) to 135° C. (275° F.).

8. The method according to claim 7, wherein said personal protection equipment includes N95 respirators.

9. The method according to claim 1, wherein said steam sterilizer is operable in a sterilization mode to sterilize articles in the chamber by regulating the temperature in the chamber to a predetermined exposure temperature in a range from 121° C. (250° F.) to 135° C. (275° F.).

10. A steam sterilizer comprising:
a chamber for receiving articles for reprocessing;
a jacket surrounding the chamber;
a first valve controlling fluid connection of the chamber to a source of steam;
a second valve controlling fluid connection of the chamber to a vacuum source; and
a control system for operating in a decontamination mode, wherein temperature of the chamber is regulated to a predetermined exposure temperature and a predetermined exposure pressure for a predetermined exposure time to effect decontamination of the articles therein, wherein the predetermined exposure temperature is in a range from 50° C. (122° F.) to 100° C. (212° F.).

11. The steam sterilizer according to claim 10, wherein said predetermined exposure pressure is in a range from 0 inHg to 29.92 inHg.

12. The steam sterilizer according to claim 10, wherein said control system is programmed to determine if the temperature in the chamber is at or above a cutoff temperature; and
if the temperature in the chamber is at or above the cutoff temperature, then operating the sterilizer in a cooling phase to reduce the temperature in the chamber prior to decontamination of the articles.

13. The steam sterilizer according to claim 12, wherein the cutoff temperature is about 70° C.

14. The steam sterilizer according to claim 12, wherein said control system is programmed to determine if the temperature in the chamber is at or above a cutoff temperature; and
if the temperature in the chamber is below the cutoff temperature, then operating the sterilizer in a heating phase to increase the temperature in the chamber prior to decontamination of the articles.

15. The steam sterilizer according to claim 14, wherein said control system increases the temperature in the chamber in the heating phase by pulsing steam into the chamber.

16. The steam sterilizer according claim 10, wherein said predetermined exposure time is about 30 minutes.

17. The steam sterilizer according to claim 10, wherein said articles include personal protection equipment (PPE) made of material sensitive to high processing temperatures in a range of 121° C. (250° F.) to 135° C. (275° F.).

18. The steam sterilizer according to claim 10, wherein said control system is programmed to operate the steam sterilizer in a sterilization mode to sterilize articles in the chamber by regulating the temperature in the chamber to a predetermined exposure temperature in a range from 121° C. (250° F.) to 135° C. (275° F.).

19. A method of operating a steam sterilizer in a first operating mode wherein articles are sterilized under high temperature conditions at a temperature of at least 121° C. (250° F.) and above-atmospheric pressure, and in a second operating mode wherein articles are decontaminated under low temperature conditions at a temperature not greater than 100° C. (212° F.) and sub-atmospheric pressure.

20. The method according to claim 19, wherein the steam sterilizer transitions between the first operating mode and second operating mode by operator selection of modes.
